(19) United States
(12) Patent Application Publication
(10) Pub. No.: US 2006/0142249 A1
(43) Pub. Date: Jun. 29, 2006

(54) Ophthalmic use

(76) Inventors: Wolfgang Froestl, Basel (CH); Rudolf Markstein, Rheinfelden (DE); Katrina L Schmid, Brisbane (AU); Anne-Ulrike Trendelenburg, Weil am Rhein (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/516,246

(22) PCT Filed: Jun. 27, 2003
(86) PCT No.: PCT/EP03/06850

§ 371 Date: Oct. 4, 2005

(30) Foreign Application Priority Data
Jun. 28, 2002 (EP) 02014412.7

(51) Int. Cl. A61K 31/675

(52) U.S. Cl. 514/89

(57) ABSTRACT

The present invention relates to the use of a GABA-C-antagonist in the preparation of a medicament for the treatment of myopia.

The present invention relates to the use of a GABA-C-antagonist in the preparation of a medicament for the treatment of myopia, and to a method to treat myopia comprising administering an effective amount of a GABA-C-antagonist to a patient in need of said treatment.

As used herein, treatment of myopia refers in particular to the control of the abnormal axial growth of the eye, more particular to control development of myopia (nearsightedness), in particular to stop and/or prevent development of myopia.

U.S. Pat. No. 5,385,939 (Laties & Stone) discloses a method for regulating the axial growth on an animal's eye, comprising the administration of an effective amount of 2-hydroxy-saclofen, a GABA-B antagonist. Other GABA-B-antagonists such as phaclophen, 5-aminovaleric acid, 3-aminopropyl (diethoxymethyl) phosphinic acid, 3-aminopropyl (n-hexyl) phosphinic acid, and 3-aminopropyl phosphonic acid are specifically mentioned as suitable GABA-B-antagonists.

WO 98/58939 describes TPMPA as a selective GABA-C antagonist, which shall exhibit cognitive enhancing activity.

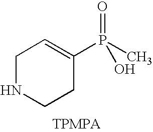

U.S. Pat. No. 5,627,169 describes a selective GABA-RHO receptor antagonist, namely TPMPA, which may play a significant role in visual processing.

Surprisingly, it has now been found that a GABA-C receptor antagonist, such as TPMPA, has a useful efficacy in the treatment of myopia.

Accordingly, in one aspect the present invention provides a method of treating myopia in an individual in need of such treatment, comprising the step of administering an effective amount of a GABA-C receptor antagonist to said individual.

The invention also pertains to the use of a GABA-C receptor antagonist in the manufacture of a medicament for the treatment of myopia.

In a preferred aspect of this invention, a GABA-C receptor antagonist should preferably be substantially inactive with respect to any efficacy for the receptor selected from GABA-B.

In another preferred aspect of this invention, a GABA-C receptor antagonist should preferably be substantially inactive with respect to any efficacy for the receptors selected from GABA-A and GABA-B.

As used herein, substantially inactive, refers to an IC₅₀ value being typically above 50 micromolar, preferably above 100 micromolar, more preferably above 250 micromolar and in particular above 500 micromolar.

As used herein, a selective GABA-C receptor antagonist has typically an IC₅₀-value being less than 40 micromolar, preferably less than 10 micromolar, more preferably less than 1 micromolar, and in particular in the range of 1-0.0001 micromolar, and more particular from 0.1-0.001 micromolar.

GABA-C antagonists suitable to treat myopia are for example represented by general formula I or general formula II, 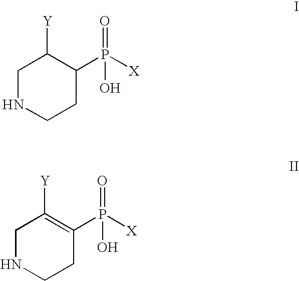
wherein X represents hydrogen, an alkyl group optionally substituted with a halogen, or a hydroxyalkyl group, and
Y represents hydrogen, a halogen, or an alkyl, alkenyl, alkynyl or acyl group, optionally substituted with halogen, nitrile, or NO₂.

In general formula 1, Y may also be an alkoxy group, optionally substituted with halogen, nitrile or NO₂.

Alkyl has up to 20 carbon atoms and may be straight-chained or branched. Alkyl is preferably lower alkyl having up to 8 carbon atoms, especially up to 4, and more especially up to 2, carbon atoms. Suitable examples include dodecyl, octyl, hexyl, pentyl, butyl, propyl, ethyl, methyl, 2-propyl, 2-butyl and 3-pentyl.

Alkenyl has from 2 to 20 carbon atoms and may be linear or branched. Alkenyl is especially lower alkenyl having from 2 to 8 carbon atoms, preferably from 2 to 6 carbon atoms and especially from 2 to 4 carbon atoms. Examples of alkenyl are vinyl, allyl, 1-propen-2-yl, 1-buten-2- or -3- or -4-yl, 2-buten-3-yl, and the isomers of pentenyl, hexenyl and octenyl.

Alkynyl has from 2 to 20 carbon atoms and may be linear or branched. Alkynyl is especially lower alkynyl having from 2 to 8 carbon atoms, preferably from 2 to 6 carbon atoms and especially from 2 to 4 carbon atoms. Examples of alkynyl are ethynyl, propargyl, 1-butyn-1-, -3- or -4-yl, and the isomers of pentynyl, hexynyl and octynyl.

Halogen is especially fluorine, chlorine, bromine or iodine, preferably fluorine, chlorine or bromine and more especially fluorine or chlorine.

Alkoxy has up to 20 carbon atoms and is preferably lower alkoxy. Lower alkoxy has up to 8 carbon atoms, preferably up to 6 carbon atoms, and is, for example, methoxy, ethoxy, propoxy, butoxy, tert-butoxy or hexyloxy.

Acyl has up to 20 carbon atoms and may be straight-chain or branched, preferably lower acyl having up to 8 carbon atoms, especially up to 4, and more especially up to 2, carbon atoms, and is for example acetyl.

Accordingly, in another aspect the invention provides the use of a compound having GABA-C antagonist activity, being in particular selected from the group of formula I and formula II as defined above, in the manufacture of a medicament for the treatment of myopia.

Thus in a yet further aspect the invention provides a method for the treatment of myopia, comprising the administration of a compound selected from the group of formula I and formula II as defined above to a patient in need of such treatment.

Examples of suitable GABA-C antagonists and their receptor profile are indicated infra for the purpose of illustration.

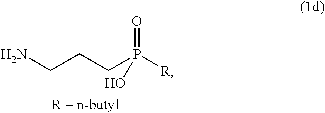

Receptor profile of representative compounds:

| | | GABA-C |
|---|---|---|
| Compound | GABA-B (micromolar) | (micromolar) |
| TPMPA | ˜500 | 2.1 |
| | agonist | antagonist |
| 1d | 38 | 62 |
| | antagonist | antagonist |

For the above mentioned indication, the appropriate dosage will of course vary depending upon, for example, the compound employed, the host, the mode of administration and the severity of the condition being treated. However, in general, satisfactory results in animals are indicated to be obtained at a daily dosage of from about 0.1 to about 10 mg/kg animal body weight. In larger mammals, for example humans, an indicated daily dosage may be in the range from about 5 to about 200 mg, preferably about 10 to about 100 mg of the compound. An administration may conveniently be administered in divided doses up to 4 times a day or in sustained release form.

The compounds used in the invention may be administered in free form or in pharmaceutically acceptable salt form, provided said compound is able to form a salt. Such salts may be prepared in conventional manner and exhibit typically the same order of activity as the free compounds.

Compounds according to the invention may be administered by any conventional route, for example intravitreally, e.g. in form of injectable solutions or suspensions, enterally, preferably orally, e.g. In the form of tablets or capsules, topically, e.g. ophthalmically, e.g. In the form of eye drops, gels, or ointments.

Accordingly, in another aspect the invention relates to an ophthalmic composition comprising an effective amount of a GABA-C antagonist and a carrier, which composition is suitable for topical ocular administration.

A carrier is typically adapted for topical administration, and is for example water, mixtures of water and water-miscible solvents, such as C₁- to C₇-alkanols, vegetable oils or mineral oils comprising from 0.5 to 5% by weight hydroxyethylcellulose, ethyl oleate, carboxymethyl-cellulose, polyvinyl-pyrrolidone and other non-toxic water-soluble polymers for ophthalmic uses, such as, for example, cellulose derivatives, such as methylcellulose, alkali metal salts of carboxy-methylcellulose, hydroxymethylcellulose, hydroxyethylcellulose, methylhydroxypropyl-cellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers.

Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof. A highly preferred carrier is water. The concentration of the carrier is, for example, from 1 to 100000 times the concentration of the active ingredient.

1-5. (canceled)

6. A method to treat myopia comprising administering an effective amount of a GABA-C receptor antagonist to a patient in need of said treatment.

7. The method of claim 6, wherein said GABA-C receptor antagonist is administered intravitreally.

8. The method of claim 6, wherein said GABA-C receptor antagonist is selected from a compound represented by general formula I and general formula II, 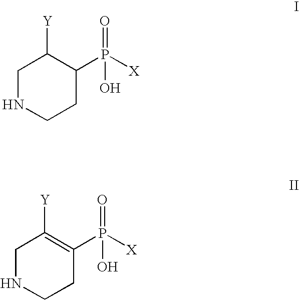
wherein X represents hydrogen, an alkyl group optionally substituted with a halogen, or a hydroxyalkyl group, and
Y represents hydrogen, a halogen, or an alkyl, alkenyl, alkynyl or acyl group, optionally substituted with halogen, nitrile, or NO₂, and
in general formula 1, Y is also an alkoxy group, optionally substituted with halogen, nitrile or NO₂.

9. The method of claim 6, wherein said GABA-C receptor antagonist is TPMPA.

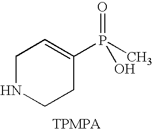

10. The method of claim 6, wherein said GABA-C receptor antagonist is compound 1d:

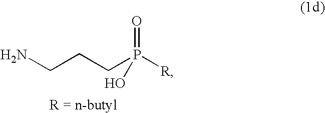

11. The method of claim 6, wherein said GABA-C receptor antagonist is administered orally.

12. The method of claim 6, wherein said GABA-C receptor antagonist is administered topically.
